(19) United States
(12) Patent Application Publication
(10) Pub. No.: US 2010/0051035 A1
(43) Pub. Date: Mar. 4, 2010

(54) LOW-PRESSURE AIRWAY MANAGEMENT DEVICE WITH ACTIVE COATING AND METHOD FOR PATIENT CARE

(76) Inventors: Shawn E. Jenkins, Duluth, GA (US); Scott M. Teixeira, Cumming, GA (US); Edward B. Madsen, Cumming, GA (US); Douglas R. Hoffman, Greenville, WI (US); Kelly D. Arehart, Roswell, GA (US); Molly K. Smith, Atlanta, GA (US)

Correspondence Address: KIMBERLY-CLARK WORLDWIDE, INC.;Tara Pohlkotte, 401 NORTH LAKE STREET, NEENAH, WI, 54956, US

(21) Appl. No.: 12/201,792

(22) Filed: Aug. 29, 2008

(51) Int. Cl. A61M 16/04

(52) U.S. Cl. 128/207.15; 604/265; 128/898

(57) ABSTRACT

An improved endotracheal tube that has a modified surface coating that can elute active agents is described. The coated surface and the use of the device according to a new method or regime for a healthcare worker to care for patients can help reduce the likelihood of ventilator acquire pneumonia or other infections in a patient's respiratory passages.

### FIELD OF INVENTION

The present invention relates to a medical device that can be employed in patient airway management.

### BACKGROUND

Patients requiring artificial respiration are often intubated with an endotracheal (ET) tube. As illustrated in FIG. 1, the tube is inserted into a patient's trachea in order to ensure that the airway is not closed off and that air is able to reach the lungs. The endotracheal tube is regarded as the most reliable available method for protecting a patient's airway.

An ET tube is composed of two primary components: a hollow cannula or catheter (i.e., the tube), through which respiratory gasses pass, and a cuff, which serves to hold the catheter in place in the trachea, prevent air from bypassing the tube and, to a limited extent, prevent contaminants from entering the lower respiratory tract. The endotracheal tube functions as an artificial airway, allowing healthcare providers to mechanically ventilate the patient. While the ET tube serves a vital role in allowing an otherwise compromised patient to breath, the ET tube, by its nature, also inhibits or bypasses many of the body's natural defense mechanisms (e.g., the ciliated epithelium and the cough or gag reflex) that prevent pathogens or other contaminants from invading and colonizing the lower respiratory tract. Moreover, indwelling devices such as endotracheal tubes often develop a biofilm, a layer of highly concentrated bacterial colonies on the surface of the device. As a result, patients with indwelling endotracheal tubes often can contract pneumonia. Such cases of pneumonia are typically referred to as Ventilator Associated Pneumonia or VAP. While certain endotracheal tube designs exist currently that attempt to prevent aspiration of contaminated secretions and/or VAP, the clinical results from the use of such designs have shown that improvement is needed. An opportunity exists to create an ET tube that combats the passage of pathogens to the lower respiratory tract by means of a multifunctional design approach.

Biofilms arise when microbes agglomerate on the surface of a substance, colonize and grow into a film. A number of naturally occurring biofilms exist, including for instance tooth plaque. The problem arises, however, when the microbial source is deleterious to humans in either concentration or intrinsic pathogenicity. For instance, films can form on items like catheters that are inserted into the human body and other medical devices used in health care settings. Biofilms have significantly different pathology as compared to non-biofilm colonies and are resistant to most currently available antibiotic treatments. This situation can leave already immuno-compromised patients susceptible to infection.

Currently some conventional ET tubes have a small second lumen with an opening situated right above the inflatable cuff, which can be used for suction of the nasopharngeal area and above the cuff to aid extubation (removal). This permits a suction system to be connected to the ET tube to allow for suctioning of secretions which sit above the cuff. Other methods involve inserting a suction catheter into the patient's pharyngeal cavity, outside the ET tube, targeting the lower pharynx. Subglottic suctioning has been shown to reduce the risk of chest infections in intubated patients. However, with current practice, a nurse or other healthcare provider will need to suction the patient over a regular interval (e.g., within about every four, six, or eight hours). This can be quite demanding on the patient as well as the healthcare provider. To reduce the burden on caregivers, continuous suctioning has also been attempted. However, risks associated with drying or damaging delicate mucosal membranes have contributed to a lack of widespread adoption. An ET tube that 1) exhibits an ability to kill or neutralize bacteria in subglottic secretions and 2) allows for longer time interval between suctioning or allows less subglottic secretion leakage over equivalent time intervals would be an improvement to the technology that caregivers would appreciate, and be beneficial to patients.

### SUMMARY OF THE INVENTION

The present invention pertains to an improved a tracheal ventilating device that has a modified surface coating. The coated surface and the use of the device according to a new regime for a healthcare worker to care for patients can help reduce the likelihood of infections in a patient's respiratory passages.

The invention according to one aspect pertains to an endotracheal tube or tracheal cannula which obturates the trachea as hermetically as possible for ventilating a patient. The present invention can combine subglottic suctioning with an advanced sealing balloon to slow the leakage rate of accumulated fluids. The better sealing balloon and slower leakage rate permits a longer dwelling or increased resonance time. Two practical applications of longer duration between each suctioning and cleaning event are: 1) more time for the fluid to be removed (i.e., suctioned) from above an ET tube cuff before being aspirated by the patient if intermittent suctioning is being used, or 2) less aggressive suctioning can be employed if continuous suction is being used.

The device has a cuffed balloon or bladder which blocks the trachea below the glottis and through which a ventilating cannula is passed, and the cuffed balloon when filled or inflated and freely unfolded without any limitation, being larger than when placed in the trachea in an inflated state, and being made from a soft flexible foil material. The endotracheal tube can have either an eluting or erodable coating to prevent the development of bacterial or other pathogenic colonies in the airway of an intubated patient. The eluting coating may also serve as a means to deliver other active medicinal or treatment agents. The eluting or erodable coating may be on an outside surface of the hollow tubing, upstream from the sealing balloon cuff, or it may be part of the outer layer of the balloon as well. The cuffed balloon has a soft, flexible foil material that enfolds over against itself and the tracheal side walls. According to the invention, when the film of the bladder is enfolded against itself, each of the lacunae or eyelets formed by the fold is significantly smaller in diameter and cross-sectional area than other folds in conventional balloon membranes, hence retarding any leakage of fluids from the area above the cuff to the lower respiratory system below. This significantly slower rate affords caregivers a longer interval between each suctioning and cleaning session. In addition, a longer interval allows the active agents in the coating to work for a longer period to kill or neutralize any harmful bacteria or other pathogens that may accumulate in pooled fluids. Hence, even if some of the fluids leak past the balloon cuff, the potential of causing an infection is reduced, thus resulting in an unexpected synergistic effect in the degree to which pathogens are prevented from entering the lower airway.

In another aspect, the present invention also pertains to a method of caring for an intubated patient with long duration indwelling ventilating devices. The method entails: providing an endotracheal tube, such as described above, having an inflatable cuff bladder with an eluting or erodable active coating for delivering a medicinal or antimicrobial agent; applying the endotracheal tube to a patient and inflating said cuff bladder to a pressure of between about 10 cm H₂O to about 30 cm H₂O; letting the endotracheal tube remain inflated within the patient for a period of more than about 30-40 minutes; and either removing or not removing (i.e., let dwell) accumulated fluids from a zone in the patient's respiratory passage above said cuff bladder at maximum once within a period of at least one hour; however, this also may including no suctioning during the period in-dwelling. The method manifests a synergy of a good sealing balloon cuff mechanism and an eluting coating on the extra-luminal surface of the ET tube, working together to minimize for the patient incidences of VAP. The device allows a caregiver a longer dwelling time for secretions between each suctioning cleaning, and/or resonance times of fluid, located above or “up stream” from the inflatable balloon, of more than about 30 minutes, which exposes any harmful microbes to active antimicrobial or therapeutic reagents eluting from the coating. Typically, the period can be as long as about two hours or more, which can lessen stress on both the patient and caregiver from suctioning and cleaning.

In another aspect, the invention relates a method of instructing caregivers on the use of an airway management device according to the present invention. The method involves: providing instruction to a healthcare worker on the use of an endotracheal tube with an inflatable cuff bladder in which at least a portion of the endotracheal tube or cuff bladder has an active coating for delivering a medicinal or antimicrobial agent, and a cuff inflation pressure of between about 10 cm H₂O to about 30 cm H₂O for a predetermined in-dwelling duration of at least about 120 minutes or more (e.g., 150, 180, 240 minutes), and either removing or not removing accumulated fluids from a zone in said patient's respiratory passage above the cuff bladder at maximum once within a period of at least two hours.

It is understood that both the foregoing summary and the following detailed description and examples are merely representative of the invention, and are intended to provide an overview for understanding the invention. Additional features and advantages of the present diagnostic indicator device and associated articles of manufacture and methods will be disclosed in the following detailed description.

### BRIEF DESCRIPTION OF THE FIGURES

FIG. 1 is a general diagram in cross-section of an endotracheal tube that is inserted through a patient's oral cavity into the patient's upper respiratory passage.

FIG. 2 is a graph depicting the difference in relative long-term capabilities for dwelling in patient of an endotracheal tube with a cuff bladder according to the present invention, and an endotracheal tube of a comparative design, within a zone of acceptable cuff inflation pressures that are do not damage the tissues.

FIG. 3 is a graph showing the relative leakage rates over a range of cuff inflation pressures from about 10 cm H₂O to about 30 cm H₂O, for the ET tube sealing technology of the present invention and three commercially available, comparative examples: A, B, and C. As the Figure illustrates, the present inventive ET tube sealing technology exhibits the lowest leakage rate of the examples, and virtually none at an inflation pressure of about 20 cm H₂O or greater.

FIG. 4 is a schematic diagram of a testing apparatus, and summarizes factors in calculating leakage rate.

FIG. 5 is a graph that summarizes the antimicrobial effectiveness of an ET tube that is coated with an active reagent, as representative of the present invention. With longer contact times, one observes a greater reduction of bacterial colony forming units (CFU/ml) in the surrounding fluid. Depending on the concentration of active reagents and initial microbe population, the present approach can reduce CFU/ml to near undetectable levels, if not eliminate them completely.

FIG. 6 is another illustration of the synergistic effect of using an eluting active coating in combination with an improved cuff sealing technology, according to the present invention, to produce better antimicrobial performance over time. The combination reduced CFU/ml as a result of contact with the active agents on the surface and within the folds of the inflated cuff balloon.

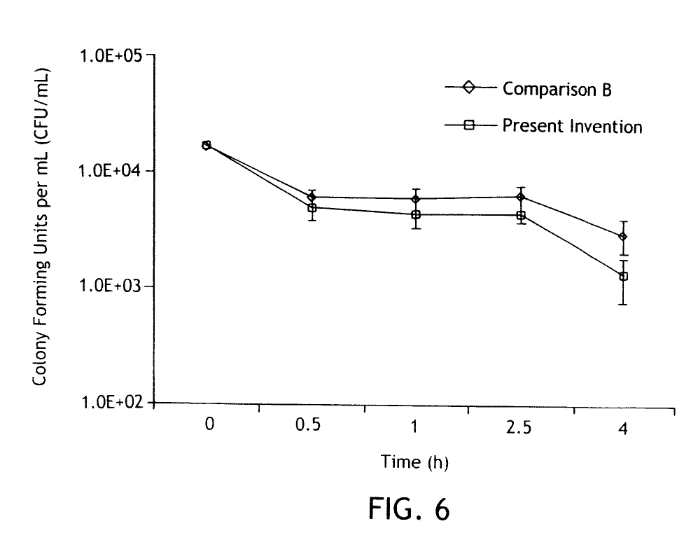

FIG. 7 is a micrograph showing a section of an inflated cuff balloon against a curved sidewall forming a relatively tight fit between the two. The image shows that the film or foil membrane of the cuff folds tightly against itself and creates a minimal capillary pore (lacunae), which can reduce the rate of leakage of any accumulated secretions.

### DETAILED DESCRIPTION OF THE INVENTION

### Section I.—Description

The present invention employs a multifunctional design approach that combines two or more functionalities to bring forth a synergistic effect to prevent aspiration of contaminated secretions, reduce colonization of the lower respiratory tract, and/or ultimately reduce the incidence of VAP. Specifically, by bringing together the features of an improved sealing cuff technology with subglottic suctioning and/or an eluting surface coating, one can achieve synergistic benefits in patient care and preventing aspiration of contaminated secretions. The invention, according to certain embodiments, builds upon the design and features of previous endotracheal tube designs, such as that described in U.S. Pat. Nos. 6,526,977 and 6,802,317, to F. Göbel, the contents of which are incorporated herein by reference. F. Göbel describes in the patents a tracheal tube having a cuffed balloon that obturates a patient's trachea as hermetically as possible. Attached to an air tube, the cuffed balloon blocks the trachea below the patient's glottis, and is sized to be larger than the tracheal diameter when in a fully inflated state. When inflated in the patient's trachea, the larger size leads the balloon to enfold upon itself, creating a capillary-sized convolution, which arrests free-flow of secretions across the balloon seal. The particular features of the cuffed balloon permits one to seal tightly against the tracheal wall while inflicting minimal damage to the tissues.

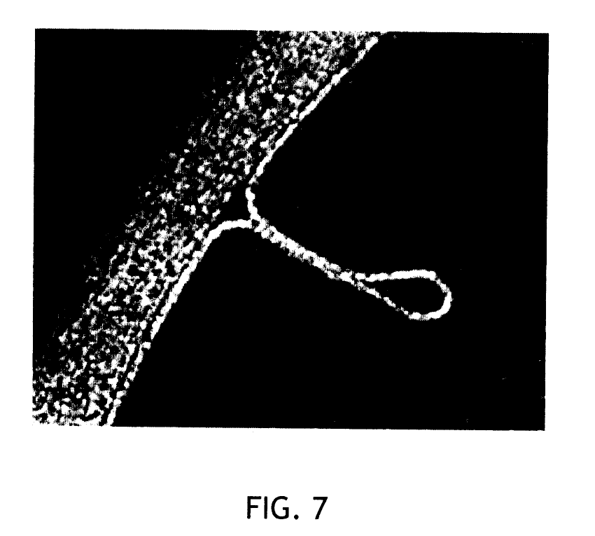

The present invention combines improved sealing with suctioning above the ET cuff and allows for reduced leakage or of secretions to the lower trachea. Further the ability to have smaller capillary pores (lacunae) or folds, such as shown in FIG. 7, with larger wall surface area allows any antimicrobial agent that is on the surface of the cuff membrane to react with the fluid secretions. Utilizing the tight seal of the cuffed balloon against the walls of the trachea can prevent or reduce leakage of secretions and other fluids past the cuff. A significant slowing or retarding in the rate at which leakage occurs can extend the time period between when a health care worker needs to suction the patient. A longer interval between administrating suction provides an advantage. The combination of improved seal and suction capability would conceivably allow for intermediate suction to take place without additional risk of leakage. In using intermediate suctioning, tracheal complications can be reduced. Likewise, if continuous suctioning were to be employed, a lesser rate of suction could be used again reducing tracheal complications. Furthermore, a combination of a tight cuff seal with an eluting antimicrobial coating, for example, would conceivably permit the eluting coating to interact with the pathogens to a greater degree over a longer duration before leakage past the cuff occurs. During this dwell time the eluting coating can deliver an antibacterial or antiseptic agent to kill pathogenic organisms. Thus, if any secretions do happen to leak down into the lungs, the fluid would contain a less concentrated amount of live or active pathogens, or be less prone to cause VAP.

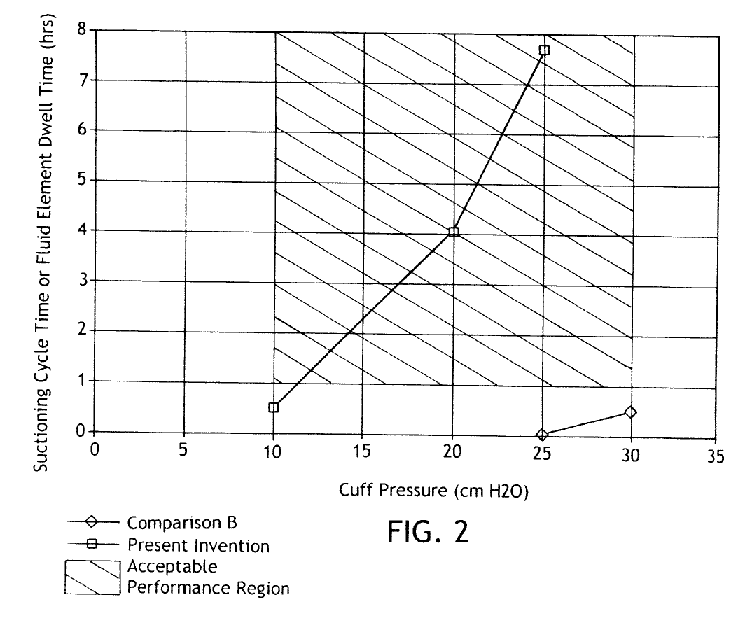

FIG. 2 quantifies the average time a caregiver has to suction a fluid element from above the cuff once it has pooled when a range of cuff pressures are used. Overlaid onto this graph is a “safe” or “acceptable” operation zone or acceptable performance region. This acceptable performance region typically is defined by the bladder or sealing cuff of endotracheal tubes being inflated to a pressure from between about 10 cm-H₂O to about 30 cm-H₂O (abscissa minimum and maximum, along the x-axis). If the pressure within the cuff balloon is too low, the sealing cuff typically does not seal well against the tracheal tissues; whereas if the pressure is too high, the cuff may cause damage to the tissues. A pressure of about 10 cm-H₂O to 30 cm-H₂O is a typical range of accepted cuff pressure. “Suctioning Cycle Time” or “Fluid Element Dwell Time” represents the calculated average amount of time a fluid element takes, once it has entered into a pool of subglottic secretions, to pass into the channels of the cuff. This calculation is based on in vitro experiments measuring rate of leakage, conducted as described in the Examples. With respect to “Suctioning Cycle Time”, times greater than 1 hour but less than 8 hours (ordinate minimum and maximum, along the y-axis) have been chosen as “acceptable.” Anything less than the former would be over burdensome to the care giver, while anything more than the latter represents a patient going a full shift without being suctions, which is atypical in practice. As one can see from the performance curves of the competitor and the current Microcuff ET tube, the Microcuff operates more within the Acceptable Performance Region. With respect to “Fluid Element Dwell Time”, note that the average time a care giver has to suction a fluid element from the secretion pool is equivalent in theory to the amount of time that fluid element dwells above the cuff.

The graph shows the relationship between the relative cuff pressure and the cycle time between each suctioning of the patient (i.e., fluid dwell time), with respect to an example of the present invention, and a competitive comparative example. As one can see the present invention enables one to seal the cuff at a pressure that is within biological tolerances for a prolonged period between each suctioning cycle as compared to currently available subglottic suctioning technologies. The present invention can effectively seal and prevent fluid leakage from above the cuff to the lower respiratory passages for more than 30 minutes at acceptable performance levels while still maintaining the pressure of the cuff within the safe zone. As the graph shows, the present invention enables a healthcare provider to extend the period between suctioning the patient for more than 30 minutes by preventing fluid leaks at acceptable cuff pressures. In typical use, the present invention affords an extended dwelling time of at least about 40 or 45 minutes at about 10 cm-H₂O, but the longer the duration the more desirable. For instance at a pressure of about 11 or 12 cm-H₂O, one can have a dwelling time of up to about 1 hour, while typically in use a pressure from about 15 cm-H₂O will permit the duration to length to about 2 hours, at about 20 cm-H₂O lengths the duration to about 4 hours, up to about 8 hours or more at about 25 cm-H₂O. In contrast, at a pressure within the range of the acceptable region, the comparative example can prevent leaking of any accumulated fluids above the cuff for up to only about 30 minutes. Hence, any prolonged period of over about 30 minutes can not be achieved without applying a pressure level beyond of the upper limit (˜30 cm-H₂0) of the acceptable region, which can damage the tracheal tissues.

The present invention is a medical device that can be used as part of an airway management system. The device includes: a balloon cuff that obturates a patient's trachea below said patient's glottis, and an air tube, the balloon cuff being attached to the air tube. The cuff may have a size larger than a tracheal diameter when in a fully inflated state and be made of a soft, flexible membrane material. Comparatively, in some embodiments the cuff may be of a design that inflates as cuff pressure is increased by stretching and thinning of the cuff wall. The air tube, alone or in combination with the balloon cuff may have at least a portion or region (e.g., upper surfaces or within the folds of the inflated balloon film) with either an eluting or erodable surface coating proximal to the cuff that serves as a controlled-release reservoir of antimicrobial, antibiotic or antiseptic agents or therapeutic agents. The balloon cuff when used in the patient's trachea can retard leakage of a volume of fluid from a zone above the cuff to the patient's lower respiratory passages for a period of more than about 30-40 minutes at a cuff inflation pressure of between about 10 cm H₂O to about 30 cm H₂O. The cuff is adapted to retard leakage of a volume of fluid from a zone above said cuff to said patient's lower respiratory passages for at least one hour. In some embodiments, leakage can be slowed for 2-3 hours at an inflation pressure of less than about 20 cm H₂O, and when necessary any leakage can be further retarded for a period of over 3 hours at an inflation pressure of no greater than about 25-28 cm H₂O. In certain embodiments, the air tube and said cuff balloon each has a surface with an erodable coating.

Application of a product that has continuous suctioning can cause the trachea to dry out and damage the tissues of the airway, even at a typical cuff pressure of about 25-30 cm H₂O pressure. With the present device one does not need to apply as great an amount of suction pressure because the leakage rate is slower than that associated with conventional endotracheal tubes. This reduces the risk of tracheal tissue damage due to drying. Moreover, the increase in dwelling time proximal to the cuff allows the antibiotic or antimicrobial chemistry of the eluting coating to interact longer with pathogens and kill more of them more thoroughly. In addition, when not employing continuous suctioning but rather discrete cleaning is used, this permits a longer interval between individual suctioning events.

### A. Endotracheal Tube

We have found that the present invention exhibits certain synergistic benefits that are not described or appreciated by others. The present invention involves a combination of an improved sealing cuff technology with subglottic suctioning, a combination of an improved sealing cuff technology with eluting or erodable surface coatings, or a combination of all three elements.

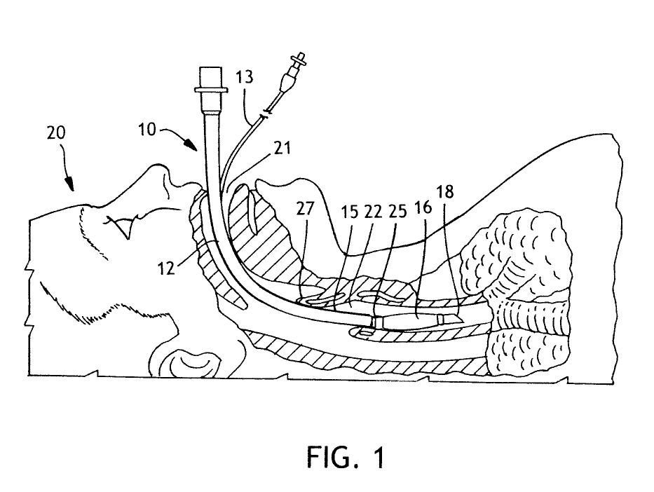

FIG. 1, depicts various elements of an ET tube 10 placed in a supine patient 20. The ET tube is inserted through the oral cavity 21, into the trachea 22, past the gottal flap 27. For purposes of illustration, the present invention involves, in part, a modification of the surface of the ET tube 10 (catheter 13 and/or balloon) with an eluting coating 15 that serves as a reservoir for reactive agents (e.g., antimicrobial or, or therapeutic), which can be released in a controlled fashion over time into the localize environment immediately proximate to the ET tube in a patient's respiratory tract 25. The ET tube has an inflatable balloon or bladder 16 attached co-axially around a hollow conduit 18, or cannula 4; an eluting surface coating which provides an extended-time controlled-release source for antimicrobial reactive or therapeutic agents and is situated over at least part of a surface of the bladder, the bladder being adapted to inflate restrictively within a respiratory channel 25 so that any fluid flowing through the channel, but external to said hollow conduit, fully interacts with the reactive or therapeutic agents. The eluting surface coating 15 covers at least part of an outer surface of said hollow conduit. In one embodiment, at least part of the hollow conduit or the bladder has a coating that forms an erodable surface when exposed for a predetermined period in an aqueous environment.

In another embodiment pertaining to the improved sealing cuff technology applied, the cuff bladder has a large surface area in its inflated state within the respiratory channel. The cuff bladder is not totally distended, but fold in upon itself tightly. Any eyelets or lacunae formed by the folds are small in diameter, not more than about 100 or 150 microns. The folds have an average surface area to volume ratio of >150 l/cm. Typically, the cuff balloon has an average surface area to volume ratio of ≧160 l/cm. A large surface area allow the cuff to be coated with more eluting coating and more surface to interact with per unit volume of liquid. This feature and better sealing cuff results in an increased contact time for the fluid to interact being allow the antimicrobial reactive agents to achieve greater efficacy of killing pathogens that may be in the fluid. The fluid contact times are more than 30 or 40 minutes, against a surface coated with an eluting antimicrobial or other surface reactive or therapeutic coating. Typically, this contact time can exceed 1 or 2 hours. The surface and fluid being located above or “up stream” from the inflatable balloon, one does not need as much loading of reactive agent on the surface of the device, or alternatively one can have a lower elution rate.

This invention involves a multifunctional approach to designing an endotracheal tube, resulting in synergies that prevent aspiration of contaminated secretions into a patient's lower airway. Specifically, the design features of interest involve a better sealing cuff design, the application of an eluting coating, and the capability to suction contaminated secretions that have pooled above the cuff. In general it is accepted that leakage of fluids past a cuff occurs through channels that form by the cuff wall folding back on itself within the confined space of the trachea. These channels allow fluid to pass the cuff. A cuff that provides a better seal and restricts flow of fluids past the cuff would prevent contaminated fluids from reaching the lower respiratory tract or at least slow its occurrence. Use of a micro-thin cuff technology is one method to accomplish this result. The employment of micro-thin cuff technology results in smaller channels when the cuff wall folds back onto itself. The smaller channels restrict flow of fluids past the cuff better than standard materials. In doing so, the risk of contracting a VAP is conceivably be reduced. The use of eluting antimicrobial coatings to prevent infections is common in medical devices such as Foley urinary catheters and intravenous catheters. Coatings conceivably prevent or delay device colonization and ultimately biofilm formation on the device. It is well recognized that, upon biofilm formation, the device becomes a persistent source of infection that is not treatable with systemic antibiotics. Eluting antimicrobial coatings can “neutralize” pathogens that come in close proximity to the coated surface via transport of body fluids or secretions. Once neutralized the pathogens no longer present a risk and the secretions become benign or at least present less of a risk if subsequently gaining access to the lower respiratory tract. Finally, the ability to suction or remove the pooled secretions from above the cuff before aspiration or leakage past the cuff can occur has been found to reduce the occurrence of VAP. This process of suctioning above the cuff is commonly referred to as subglottic suctioning. However, historical designs were found to plug and subsequently be ineffective. Furthermore, historically, continuous suction has been used in conjunction with previous designs, but can result in complications such as tissue dehydration and/or damage. In this regard, intermediate suctioning would be preferable to reduce the risk of tissue damage. There is reason to believe that combining two or more of these functionalities would result in a synergistic effect in preventing aspiration of contaminated secretions and/or VAP. Specifically this invention involves combining;
Improved cuff sealing and suctioning capability—In that improving the cuff seal prevents or delay's leakage of fluids past the cuff, the combination of improved seal and suction capability would conceivably allow for intermediate suction to take place without increased leakage. In using intermediate suctioning, tracheal complications can be reduced.
Improved cuff sealing and an eluting coating—In that improving the cuff seal prevents or delay's leakage of fluids past the cuff, the combination of improved seal and antimicrobial coatings would conceivably allow the coating to interact with the pathogens to a greater degree before leakage occurs. Thus, if leaked into the lungs, the fluid would contain fewer pathogens or be less prone to result in VAP.

According to an embodiment of the invention, the ET tube employs a low-pressure sealing cuff balloon like that described by F. Göbel, in U.S. Pat. Nos. 6,526,977 and 6,802,317. The ET tube is characterized in that the draped fold of the balloon cuff rests on the trachea and is designed in such a way that the loop formed at the dead end of a fold has a small diameter which inhibits the free flow of secretions through the loop of the fold. The cuff balloon or bladder is made of a soft, flexible membrane or foil material that forms at least one draped fold in the cuffed balloon trachea, wherein the foil has a wall thickness below or equal to 0.01 mm and the at least one draped fold has a loop formed at a dead end of the at least one draped fold, that loop having a small diameter which inhibits a free flow of secretions through the loop of the at least one draped fold.

As F. Göbel appreciated, the flow of secretions can be influenced by a specific design of the cuff folding in the area of the loop of the fold, i.e. at the base of the fold. While in the prior art it has so far been assumed that cuffed balloons with a draped fold cannot rest on the trachea in a sufficiently tight manner because of the low filling pressure, the invention shows a method of inhibiting the flow of secretion, the method being employed in the area of the loop of the fold. When the diameter of the loop is sufficiently small at the base of the fold, the free flow of secretion through the loop is inhibited. The resultant loops at the deep end of the fold can be reduced with respect to their diameter, for instance by selecting the material or the foil thickness, in such a manner that the flow of secretion is decelerated or, ideally, stopped altogether. Conventionally, leakage was regarded as a problem of pressure, so the industry believed that the higher the pressure, and more taught the balloon, the better sealing the cuff would be.

The larger surface area of the cuff enfolds upon itself forming lacunas with a capillary size. This will then result in adequate adhesion forces of the secretion on the loop and in a sufficient viscosity-dependent resistance of the secretion to reduce the flow of secretion. The flow rate in the capillary-sized loop is then smaller than the theoretically possible rate without adhesion or viscosity forces, so that a smaller amount of secretion will flow therethrough in the course of time. In an optimum case the diameter of the loop is made so small that the flow of secretion is stopped altogether. The diameter of each lacuna may be less than 0.1 or 0.05 mm in an especially advantageous development. At a value below 0.1 mm a certain inhibition of the flow rate of the secretion through the loop can already be observed. At a loop diameter of less than 0.05 mm, the flow of secretion is further decelerated and almost stopped.

The wall thickness of the foil material can be chosen to be so small that the inner radius of the developing loops is reduced at physiologically tolerated filling pressures to such an extent that the free flow of secretion is prevented. The more flexible and thinner the material is, the smaller is the diameter of the loop. The wall thickness of the foil should be slightly smaller than or equal to 0.02 mm. When the cuffed balloon is made from such a foil, the flow of secretion through the loop will already be inhibited at a standard filling pressure. In comparison, the wall thicknesses of conventional cuffed balloons predominantly range from 0.06 to 0.1 mm.

In a variant of the invention, the wall thickness of the foil is approximately 0.01 to 0.005 mm. In the case of a wall thickness ranging from 0.01 to 0.005 mm, a soft flexible foil will already inhibit the flow of secretion in a satisfactory manner and the stasis thereof will be achieved in the area of the base of the fold, respectively.

According to a preferred embodiment the membrane or foil material of the cuffed balloon may e.g. consist of polyethylene teraphthalate (PETP), low-density polyethylene (LDPE), polyvinyl chloride (PVC) or polyurethane (PU). These materials are body-tolerated and, when being processed into correspondingly thin walls, are especially suited for forming a hermetically obturating draped fold. Copolymer admixtures for modifying the characteristics of the material are possible (e.g. LDPE-EVA). The cuffed balloon possibly may be of a material which adheres to itself and the adhesion of which helps to reduce the clear diameter of a loop at the base of the fold.

In a variant of the invention, the wall thickness of the membrane foil may be thinner in the area of the draped fold than in the fold-free area directly adjacent to the tracheal mucous membrane. Folds are preferably formed in the thin-walled cuff region because the membrane can more easily be deformed in said area. The foil base can form loops of a smaller diameter because of the smaller wall thickness. In the more thick-walled cuff region between the folds the cuff coat has characteristics that are slightly more rigid so that it only rests in a rounded form on the wall of the trachea.

In a particular manner the fold walls which are opposite to each other in a fold are interconnected in the area near the base of the fold. The point of connection may be provided directly next to the forming loop so that the size of the loop is set by said point of connection to a desired diameter. Preferably, the opposite fold walls can be interconnected at the dead end of the fold to fill the loop, whereby the flow of secretion is reliably prevented. It is also possible to weld or glue the opposite fold walls of a fold to each other.

Particularly, a fold section having a variable cross-section in the depth of the fold, in which the opposite fold walls are not materially interconnected, may be adjacent in the fold to the connection portion of the opposite fold walls. The cuffed balloon can adjust itself to the trachea in size and shape via such a pre-formed draped fold having a variable fold depth, i.e. in accordance with the concept regarding the residual cuff coat.

In another embodiment, a conventional cuff of the tracheal tube may be supplemented by a second tampon balloon which directly follows the cuff to the oral side and completely fills the so-called subglottic space (space between the upper edge of the cuff and the vocal cords). The tampon balloon consists of a foil-like, extremely expandable material which closely nestles on the local structures of the subglottic space under expansion. To optimize the tamponade of the subglottic space, it is recommended that the balloon should be pre-formed in accordance with the morphology of the space to be filled. A fold-free surface of the tampon balloon which is as smooth as possible is to prevent any accumulation of secretion and the formation of a subglottic germ reservoir, respectively.

However, the expansion of such a displacement body is accompanied, above all in the region of the morphologically complicated inner larynx, with the formation of pressure peaks in the area of prominent structures that extend into the local space. When the filling pressure of the tampon balloon exceeds the perfusion pressure of the vascular bed supplying the film of the mucous membrane, serious lesions of the wall structures may ensue, above all in the region of the dorsolateral subglottic larynx.

To prevent the larynx from being damaged by pressure, it is recommended that the tampon balloon should also be provided with a residual volume, i.e., its volume in the freely unfolded state should exceed the volume of the inner larynx to be filled. The tampon balloon complies with the inventive principles governing the design of a sealing and gentle cuffed balloon. The formation of the above-described capillary-like structure is thereby prevented.

Since many applications require a reliable and mechanically loadable anchorage of the tracheal tube within the windpipe, a certain minimum wall thickness of the fixing cuff must not be exceeded, depending on the respective quality of the material. Despite a substantial reduction of the wall thickness, the formation of fluid-draining loops cannot adequately be prevented in all cases.

Nevertheless, in order to ensure optimum sealing characteristics, the present invention suggests that the mechanically fixing cuff should be supplemented by an additional sealing tampon balloon complying with the above-described principles of design that govern sealing aspects and tissue compatibility. The tampon balloon can be subjected to a minimum filling pressure of preferably 10 to 15 mbar which has only the function to unfold the thin, sealing balloon wall.

The fixing cuff can be mounted at the caudal side of the device, and the tampon balloon relative thereto at the cranial side. During intubation the fixing cuff is pushed forwards beyond the cricoid cartilage of the larynx, preferably into the region of the middle tracheal third where it is anchored in a reliable and tracheally compatible manner. The tampon balloon which is arranged at the cranial side relative thereto can expand in the direction of the subglottic space where, being arranged upstream of the fixing cuff, it obturates secretion seeping from the pharynx.

The fixing cuff and the tampon balloon may also be positioned in sequential order on the ventilating cannula. The tampon balloon while expanding to the cranial side can partly cover the so-called subglottic space up to the glottic plane or slightly beyond said plane. Since both balloons are filled separately via corresponding supply lumina mounted inside the tube shaft, the functions of fixing cuff and tampon balloon in the case of a serial arrangement can largely be controlled independently of each other. Desirably, the point of connection between the two serially arranged balloons is configured such that no secretion can accumulate in the area thereof in the filled and tracheally unfolded state. The fixing cuff can be enclosed by the tampon balloon at least in portions, preferably completely. The outer tampon balloon can thus expand to the caudal side up into a variable area of the fixing cuff. The formation of a germ reservoir between the balloons is thereby prevented.

In a preferred variant, the cuff which tracheally fixes the tube is entirely surrounded by the tampon balloon. The tampon balloon extends from the caudal end via the cranial end of the cuff into the so-called subglottic space and into the area of the vocal cord plane or slightly beyond said plane. In this interposed embodiment regarding the combination of a fixing cuff with a tampon balloon, the invention suggests a particular mode of handling. After conventional intubation the outer liquid-obturating tampon balloon is initially to be filled and is to nestle on the wall of the local space to be filled at a minimum pressure. Subsequently, the fixing cuff arranged in the interior is then unfolded in the customary manner and at the standard filling pressures for stabilizing the tube in the trachea. Hence, the fixing cuff has no fluid contact, i.e., the possible formation of loops in the coat of the inner cuff has no fluid-draining effect.

To prevent the two balloons from adhering to each other while unfolding, and to ensure their independent mechanical characteristics during ventilation, it is suggested that a small amount of a separating medium, such as oil or talcum, should be introduced into the space between the balloons.

When the tampon balloon which is subjected to a minimum pressure that is gentle on the tissue is expanded into the region of the glottis or slightly beyond said region, the potential path of entry for germ-containing secretion is extended to a maximum. The volume of secretion is reduced by the displacing tampon balloon to a small film exposed to the epithelium-inherent defense factors and is thus reduced with respect to its flow rate to a maximum degree. On the whole, the efficiency of the local defense mechanism is thereby optimized considerably. Since the stasis of germ-containing material above the tracheally fixing cuff is suppressed virtually completely, changes in the mucous membrane due to chronic inflammation can additionally be prevented. When the tampoon balloon extends beyond the vocal cords into the supraglottic region, the permanently traumatized contact of the tube shaft with the vocal cords can be reduced by the tension-free lining of the vocal cords with the coat of the tampon balloon.

Every conventional tube (high-volume/low-pressure, high-pressure/low-volume cuff or intermediate designed cuff) that does not, as preferred above, eliminate the subglottic germ reservoir can be optimized with respect to tightness and tissue compatibility by the interposed arrangement of the fixed cuff and by a tampon balloon having a wall thickness of only a few micrometers. The outer tampon balloon should only slightly exceed the fixing cuff as to its cranial and caudal extension or should dimensionally correspond to the fixing cuff. The two balloons can be filled separately. In this case, too, the outer, hermetically obturating cover which has a thickness of only a few micrometers is to prevent by way of its initial unfolding that the formation of fluid-conducting tubuli of the inner cuff with the greater wall thickness causes a leakage of secretions. Such an arrangement of a sealing and stabilizing coat or envelope makes the invention applicable not only to tracheal tubes, but also in particular to tracheal cannulas. Tracheal cannulas are inserted not via the larynx, but via a surgically laid opening (stoma) in the windpipe.

The maintenance of the filling pressure in all of the above-described cuff and tampon balloons is ensured by an extracorporeally mounted reservoir. In accordance with the Lanz principle, such reservoirs may be equipped with a self-regulating valve mechanism, or may be designed in the manner of simple valve-carrying reservoir balloons. To be able to estimate the desired filling pressure, an imprinted figure or a specific form of the reservoir balloon may be chosen which specifically varies in a corresponding filling state.

To prevent pressure variations inside the trachea or the larynx from affecting an expansion of the wall structures, the material compliance of the reservoir balloon should not exceed that of the cuff or tampon balloon. The supply legs leading to the tampon balloon should be chosen such that they have a sufficiently large lumen so as to effect a rapid pressure compensation. Any suitable fluid may be used for filling the sealing or fixing cuffed balloons. When liquids are used, a valve mechanism can be dispensed with and the filling operation can solely be controlled via an open liquid column.

The seal of the tracheal or laryngeal remaining lumen (which is created during intubation) by way of a reduction of the wall thickness of the cuff coat to the range of a few micrometers also permits the liquid-tight tamponade of the intubated trachea of neonates, babies or infants. On account of the high tissue vulnerability with respect to conventionally cuffed balloons, all kinds of sealing devices have so far been dispensed with during intubation. A tissue-compatible, liquid- or gas-tight sealing of the extremely sensitive upper airways would be possible by way of an elongated tampon balloon which fills the trachea and the larynx and is subjected to minimum pressures (preferably of 5 mbar).

In yet another embodiment, the cuff balloon may be of the low volume/low pressure type, described by P. J. Young et al, in CRITICAL CARE MED. March, 2006; 34 (3): 632-9 16505646 (P, S, E, B) A low-volume, low-pressure tracheal tube cuff reduces pulmonary aspiration.

Although others have commented that ET tube cuff designs that swell in thickness upon absorbing moisture from the surrounding tissues, such as described in U.S. Patent Publication No. 2004/0220534, (a coated medical device that can have a cuff), the present invention is distinguishable. While adding a swellable coating to the cuff may be one mechanism by which to improve seal and reduce leakage rate, one may expect that such a coating could change the mechanical properties, and likely may have a deleterious effect on sealing characteristics that cause channels or lacunae with larger diameters that one would need to fill. Nonetheless, if successful in improving the tracheal seal, one may employ such an approach for a sealing cuff with the present invention to achieve similar synergies as described herein when combined with an eluting or erodable coating, or subglottic suctioning method.

### B. Benefits to Health Care

A benefit of the present invention is that continuous suction, if employed, does not need to be as strong because the leakage rate of secretions pass the cuff is slower. Tracheal tubes and care products that employ continuous suctioning can cause the trachea to dry out can result in trauma to the tissues. Additionally when insufficient amounts of liquid are present, the thick mucus can cause the suction lumen to clog. Being able to use a lower rate of suction mitigates this risk. Moreover, for similar reasons, if periodic suctioning is employed, the interval period between each suctioning and cleaning session can be extended, which is advantageous to both the patient and caregiver. A health care provider can care for a patient over a longer interval between each cleaning under less stressful conditions. According to another aspect of the present invention, the caregiver can follow a method that involves, providing an intubated patient with a tracheal ventilating device that has a cuffed balloon or bladder constituting an enfolded film or membrane that has a coating which elutes a medicinal or antimicrobial agent. According to the method, a caregiver suctions or drains accumulated fluids at maximum once within a period of 1.5 to 2 hours, or possibly over 2 hours. The endotracheal tube and inflated cuff bladder retards or slows leakage of a volume of accumulated fluid from a zone upstream or above the cuff bladder to said patient's lower respiratory passages for at least one hour, typically for 2-3 hours at a cuff inflation pressure of less than about 20-18 cm H₂O. In certain circumstances, the endotracheal tube and inflated cuff bladder allows the caregiver to slow the leakage rate of a volume of fluids for over 3 hours. In other situations, the cuff can slow leakage rate up to about 7 hours at a cuff bladder inflation pressure of no greater than about 25-28 cm H₂O. Over these extended periods an antimicrobial, antibiotic or antiseptic, or therapeutic reagent can elute from the actives-coated surface of the endotracheal tube and cuff bladder, and interacts with and neutralizes any pathogens in the accumulated fluid.

As FIG. 2 shows, the long term dwelling capabilities of an endotracheal tube according to the present invention as compared to conventional endotracheal tube designs are significant. A fluid element located within a pool of subglottic secretions proximal to the cuff resides there over a much longer period of time with the present invention. This allows a care giver more time to remove that fluid element from the patient via subglottic suctioning or allows the care giver to be more effective at removing fluid at similar suctioning cycle frequencies. Additionally, if an eluting coating is employed, the fluid element is acted upon over a much longer period of time by the therapeutic agent before passing by the cuff as a leakage event. The longer exposure time to the therapeutic agent allows the agent to be more effective or allows the agent to be equally effective at lower surface loadings.

### C. Coatings

The endotracheal tube and cuff bladder can each have a surface coating that is either eluting or erodable in an aqueous environment. Coating technologies, designed to elute a therapeutic or bioactive agent from the surface of a coated device, are numerous and varied in their design. Often the appropriateness of a particular coating technology platform for any given application is judged by factors such as the incremental added cost for the coating, the in-use environment or area of application of the coated device, the composition of the surface or device to be coated, the duration of exposure to the environment, the targeted therapeutic level of eluting agent, as well as a myriad of other factors. For the purposes of this invention, any coating technology that elutes one or more agents into the environment over time is considered applicable. For purposes of example, but not for limiting of the invention, applicable coating technologies include those where an agent is dispersed as molecules, or agglomerates, micelles or other reservoirs of active agent within a matrix material. Often times the matrix material is an additional material, differing in composition as compared to the surface being coated. Molecular dispersal of the active agent within the matrix can take place as a solution of the therapeutic or bioactive agent within the matrix, or via a third component such as a zeolite; wherein the therapeutic agent is molecularly entrained within the third component and wherein the third component is dispersed within the matrix. Dispersal of reservoirs of active agent can take place as nano- or micro-capsules, where the capsules contain reservoirs of active agent contained within an impermanent shell. When exposed to an environment in which the eluting agent or capsule shell is, for example, soluble, the agent will elute from the matrix through diffusion and solubilization. Conversely, the coating may be exposed to an environment in which the matrix component, rather than the active, is soluble. This exposure may serve to soften, dissolve, degrade, absorb or erode the coating from the coated surface, which in turn elutes the agent. By yet other means the matrix component may be biodegradable within the environment in question, which results in the elution of the therapeutic agent or bioconversion of the matrix into a therapeutic agent. Other technology platforms include tailoring a matrix layer to have a micro- or nano-structure that allows an eluting agent efficient transport within or through its structure. In this case the micro- or nano-structured layer may have the eluting agent dissolved within its layer or may simply be a transient layer by which elution is controlled in some manner by the layer's structure. Furthermore, coating technologies exist wherein the therapeutic or bioactive agent has been compounded into a matrix material, that material being the primary component of the device or surface in question. Finally, a technology exists where elemental metal nano-particulates are attached to a surface without the need for a matrix material. In this case, the elemental metal forms a soluble layer upon exposure to the environment that results in a controlled dissolution of metal ions into the environment.

According to an embodiment of the present invention, one may incorporate a polymeric material on the endotracheal tube that is resistant to biofilm formation. The surface treatment can be incorporated to create an erodable surface, which can hinder the ability of planktonic microrganisms to attach to the surface of the medical device and transform into sessile genotypes (i.e., biofilm). An “erodable surface,” as used herein refers to a surface that biodegrades into natural metabolites and/or dissolves when exposed over prolonged periods to an aqueous environment. The gradual removal of layers of the surface coating can disrupt the formation of biofilms by eliminating a stable substrate upon which bacterial colonies can grow to a sufficiently large concentration to become deleterious to a patient's health. In other words, over a predetermined time period when exposed to an aqueous environment, layers of the erodable surface with nanometer-scale thinness slough off and deprive bacteria a stable base upon which to establish a colony for long. The polymeric material is degradable and has mechanical properties similar to those of medical devices currently in use as indwelling devices. The polymer can be prepared from a multifunctional acid and a multifunctional alcohol, and has demonstrated that it can degrade under a variety of biologically relevant conditions and may be used to deliver anti-infective agents. One may incorporate the degradable polymer feature into the material surface, thereby eliminating the very thing that a biofilm must have to form, namely, a stable, inviting substrate surface. The surface chemistry or morphology should reduce the affinity of the microbes to attach and colonize the extralumen and intralumen surfaces of the endotracheal tube.

A material appropriate for use to prevent biofilm formation may be formed by means of condensation polymerization of a polyol and a polyacid to create a crosslinked network. The polyol and polyacid combination should satisfy the requirement that one have at least difunctional groups and the other at least trifunctional groups to allow for crosslinking. Examples of polyols and polyacids may include, but are not limited to, polyethylene glycol, glycerol, maltose, maltodextran, butanetriol, xylitol, 1,2,4-butanetricarboxylic acid, 1,3,5-pentane tricarboxylic acid, citric acid, and malic, adipic, sebacic and succinic acids. The synthesis method does not require additional agents (e.g. catalysts, solvents) other than the monomers, so the material will not contain toxic or irritating components as long as the monomers chosen are biocompatible. These polymers contain ester bonds that degrade by hydrolysis, which is favored over enzymatic degradation since tissue water content is more consistent among individuals than are enzyme concentrations. Increasing or decreasing the crosslink density will slow or speed the degradation rate and make the material more rigid or flexible, respectively. The monomers utilized in making the material also can be chosen to adjust its degradation rate (more hydrophilic, faster degradation). In addition, the monomers themselves may provide a therapeutic benefit when the material degrades and they are released. For example, the acidic component could act to reduce the pH of the surrounding environment to discourage growth of infectious bacterial species. Introduction of other components into the monomer mixture, such as lactic acid, which is a natural pH regulator and relatively hydrophilic, could provide further therapeutic benefits and/or modulate the polymer degradation characteristics. In addition, other molecules such as hormones or drugs could be incorporated into the device to create a delivery system providing controlled release of these therapeutic agents. Three materials for potential use in fabricating a flexible device or device coating have been synthesized. The first material was synthesized by polycondensation of approximately 0.15 mol each of glycerol (HOCH2CH(OH)CH2OH) and sebacic acid (HOOC(CH2)8COOH). To begin, the sebacic acid is placed into a 3-neck flask that was held in a silicone oil bath at about 135° C. until the acid melts, at which point glycerol is added to the flask. The reaction mixture is maintained at this temperature and stirred under a flow of nitrogen for 20 hours. The contents of the flask are then transferred to a small metal container (which simplifies removal of the polymerized product upon reaction completion) and placed in a vacuum oven at 135° C. and −95 kPa for 30 hours to remove the water produced by the reaction and drive it to completion. The polymer yielded by this reaction is a yellowish, translucent to opaque material that is quite rubbery and flexible. A second material may be synthesized by polycondensation of 0.05 mol xylitol (HOCH2(CH(OH))3CH2OH) and 0.0825 mol sebacic acid using the same reaction conditions described above, except the mixture was stirred under nitrogen for 24 hours and held in the vacuum oven for 40 hours. For a third material, about 0.025 mol xylitol, 0.041 mol glycerol and 0.0825 mol sebacic acid were reacted under conditions identical to those used for the second material. These two materials were both transparent to translucent and flexible. Further, xylitol has been identified as a potential antimicrobial for a number of common pathogens associated with biofilm formation and there is the possibility that this additive within the matrix may also offer some antimicrobial properties to the polymer.

Another example of an erodable surface coating can be a co-extruded tube having a polyvinylchloride (PVC) core and a degradable extra- and intralumen sheet. The degradable component can be made with a homopolymer, a copolymer, or blend selected from the lactone family. A copolymer and/or blends may include a polyalkylene component, polyacrylate, polyvinylpyrrolidone, and/or a poly(ortho)ester. Other nondegradable components may also be incorporated as long as they exhibit some degree of solubility in an aqueous environment. The rate at which the surface erodes is dependent on the rate of degradation and degree of solubility of the components, thereby allowing for a controlled rate and time of protective action.

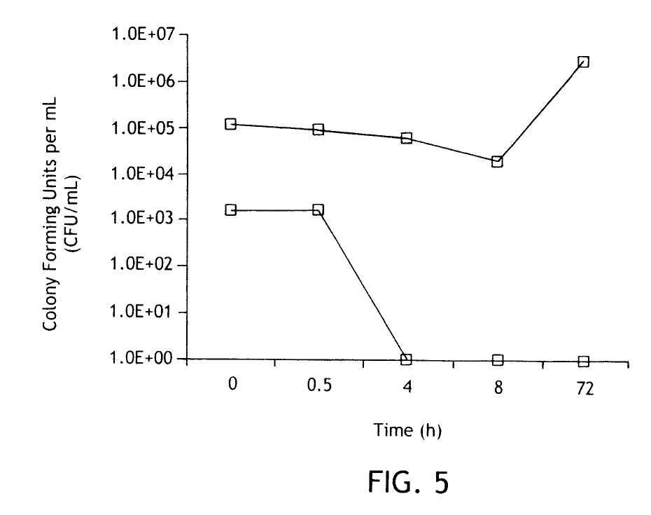

It is envisioned that given the improved sealing capacity of the balloon cuff, according to the present invention, which affords a longer dwelling time between suctioning events, a synergistic effect will manifest to neutralize the microbes or colony or colony fragments that are eroded from the surface of the medical device, since the eroded detritus will be exposed to antimicrobial agents during the dwell-time. As the results suggest in FIG. 5, biofilm forming microbial colonies (CFU/ml) can be neutralized to near undetectable levels in the fluid as long as sufficient concentration of antimicrobial agents are present. The longer microbes are exposed in the fluid to the antimicrobial agents the less they will likely be able to form biofilms.

Various additional functional reagents or “actives” may be incorporated into a coating and can demonstrate synergistic performance when applied to and eluted from a medical device, such as an ET tube of the present invention, and combined with the improved sealing cuff technology (i.e., U.S. Pat. Nos. 6,526,977 and 6,802,317). It is envisioned that different kinds of antimicrobial, antibiotic or antiseptic agents that are effective against bacteria, fungi, yeasts, viruses, parasites, etc., may be incorporated as an active agent to be eluted from the balloon or cannula surface coating when applied to the surface of an ET tube according to the present invention. These may include for instance, any one or a combination of the following antibiotic, antiseptic, preservative compounds: ample spectrum penicillins; penicillins and beta-lactamase inhibitors; cephalosporins; macrolides and lincosamines; quinolones and fluoroquinolones; carbepenems; monobactams; aminoglycosides; glycopeptides; tetracyclines; sulfonamides; rifampin; oxazolidonones; streptogramins; benzoic acids and benzoates; sorbic acids and sorbates; acetic acid and acetate salts; lactic acid; propionic acid; fumaric acid; citric acid; nitrates; parabens; sulfites; sodium chloride; phosphates; peroxides; alcohols; quaternary ammonium compounds; boric acid; chlorhexidine gluconate; iodine; mercurochrome; octenidine dihydrochloride; phenol(carbolic acid) compounds; sodium hypochlorite; aldehydes; halogen-releasing compounds; ethylene oxide; formaldehyde; anilides; biguanides; bisphenols; halophenols; and cresols. Additionally, certain treatment agents may also be included, such as antithrombotics or anticoagulants (e.g., warfarin, heparin, or aspirin). Other ingredients may include anesthetics (e.g., by inhalation—halothane, isoflurane, sevoflurane, desflurane, or gases such as ethylene, nitrous oxide and xenon; or for local anesthetics—Amino esters: benzocaine, chloroprocaine, cyclomethycaine, dimethocaine/larocaine, propoxycaine, procaine/novocaine, proparacaine, tetracaine/amethocaine; or Amino amides: articaine, bupivacaine, carticaine, cinchocaine/dibucaine, etidocaine, levobupivacaine, lidocaine/lignocaine, mepivacaine, piperocaine, prilocaine, ropivacaine, or trimecaine; or combinations thereof (e.g., lidocaine and prilocaine (EMLA)), or natural local anesthetics (e.g., saxitoxin, or tetrodotoxin).

### Section II.—Empirical Examples

To the extent that a combination of an improving the sealing efficacy of the cuff against the tracheal tissues prevents or retards leakage of secretion fluids pass the cuff and increases the contact time between pathogens and the active reagents in antimicrobial coatings helps reduce VAP, the present invention has exhibits significant benefits. In the examples, below, where the effectiveness of an antimicrobial coating are tested, the coating is elutes silver ions into the immediate environment of the ET tube. As such, an “erodable” coating may also be incorporated.

### A. Leakage Rate of Secretion Fluids Pass an ET Tube Cuff

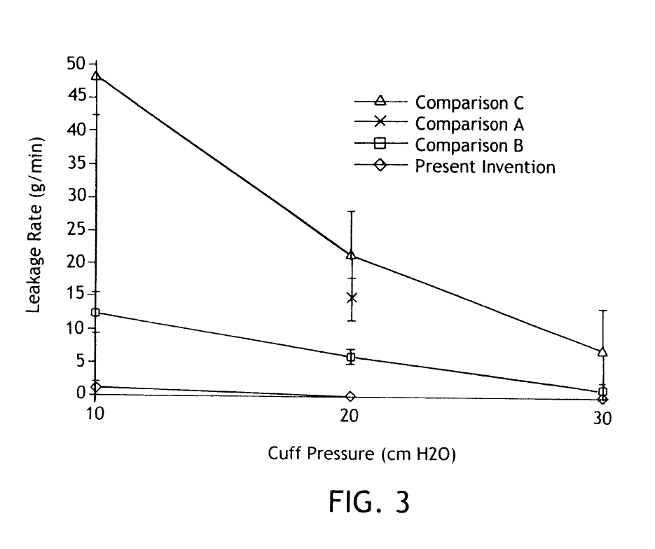

In laboratory tests we measured the approximate leakage rate of endotracheal tubes with cuff balloons for a volume of fluid that typically accumulates from secretions in an intubated patient. A 1.9 cm inner diameter hollow glass cylinder was used as a model of an average sized trachea. Water was used as model for secretion fluid, recognizing that secretions are often more viscous than water. As such, in this regard leakage rates can be interpreted as a “worse case” scenario. A 4 cm hydrostatic head of fluid was kept above the inflated cuff to model secretions pooled above the cuff. Given the geometry of the test system a 4 cm hydrostatic head resulted in approximately 11 ml's of fluid residing above the cuff. This volume of fluid can be compared with a reference, Detection of Pooled Secretions Above Endotracheal-Tube Cuffs: Value of Plain Radiographs in Sheep Cadavers and Patients. Greene, R; Thompson, S; Jantsch, H S; Teplick, R; Cullen, D J; Greene, E M; Whitman, G J; Hulka, C A; Llewellyn, H J, AJR Am. J. Roentgenol, Volume 163, Number 6, December 1994, pp.1333-7. The rate of fluid leakage passed the cuff (in ml/min) was measured as a function of cuff pressure. The results are presented in Table 1, and graphically in FIG. 3. In this case, within the present invention, a thin-membrane or foil cuff bladder technology was used as the improved cuff sealing technology. This improved cuff technology is one of several that may significantly slow the rate of leakage past the cuff. For instance, the low volume/low pressure cuff technology described by Young et al. (P. J. Young et al., in CRITICAL CARE MED. March 2006; 34 (3):632-9 16505646 (P, S, E, B) A low-volume, low-pressure tracheal tube cuff reduces pulmonary aspiration) may also be used.

TABLE 1

| Present Invetion, OD = 9.3 mm | | | | | Comparison A, OD = 9.6 mm | | | | |
|---|---|---|---|---|---|---|---|---|---|
| | Cuff Pressure (cm H2O) | | | | | Cuff Pressure (cm H2O) | | | |
| | 10 | 20 | 25* | 30 | | 10 | 20 | 25* | 30 |
| 1 | 0.290 | 0.000 | | 0.002 | 1 | N/A | 14.727 | N/A | N/A |
| 2 | 2.250 | 0.000 | | 0 | 2 | N/A | 20.458 | N/A | N/A |
| 3 | 0.130 | 0.051 | | 0 | 3 | N/A | 16.926 | N/A | N/A |
| 4 | 0.080 | 0.036 | | 0.002 | 4 | N/A | 10.390 | N/A | N/A |
| 5 | 2.010 | 0.087 | | 0.006 | 5 | N/A | 14.400 | N/A | N/A |
| AVG | 0.952 | 0.035 | 0.018 | 0.002 | AVG | N/A | 15.380 | N/A | N/A |
| Median | 0.290 | 0.036 | 0.019 | 0.002 | Median | N/A | 14.727 | N/A | N/A |
| SD | 1.081 | 0.037 | | 0.002 | SD | N/A | 3.690 | N/A | N/A |
| CI | 0.948 | 0.032 | | 0.002 | CI | N/A | 3.234 | N/A | N/A |
| Comparison B, OD = 9.7 mm | | | | | Comparison C, OD = 9.3 mm | | | | |
| | 10 | 20 | 25* | 30 | | 10 | 20 | 25* | 30 |
| 1 | 15.430 | 4.836 | | 0.070 | 1 | 50.000 | 17.005 | | 1.950 |
| 2 | 9.770 | 5.842 | | 0.300 | 2 | 57.900 | 23.545 | | 19.550 |
| 3 | 8.830 | 6.153 | | 3.280 | 3 | 40.680 | 34.020 | | 1.780 |
| 4 | 11.340 | 7.807 | | 1.520 | 4 | 42.580 | 17.189 | | 8.530 |
| 5 | 16.500 | 5.652 | | 0.440 | 5 | 50.480 | 15.395 | | 3.860 |
| AVG | 12.374 | 6.058 | 3.590 | 1.122 | AVG | 48.328 | 21.431 | 14.282 | 7.134 |
| Median | 11.340 | 5.842 | 3.141 | 0.440 | Median | 50.000 | 17.189 | 10.525 | 3.860 |
| SD | 3.420 | 1.092 | | 1.329 | SD | 6.903 | 7.697 | | 7.456 |
| CI | 2.997 | 0.958 | | 1.165 | CI | 6.050 | 6.746 | | 6.535 |

*Extrapolated values
*Extrapolated values

B. Synergies in Combining Improved Sealing Technology with Suction Capability

When compared to a conventional, or Comparsion A, B, and C, type ET tube with cuff design, the thin-membrane or foil cuff bladder technology of the present invention demonstrated the lowest rate of leakage at each cuff pressure tested between about 10 and 30 cm H2O, which defines an accepted safe inflation pressure zone. The slower rate of leakage leads to a longer fluid dwelling or “resonance” time above the cuff. Average residence or resonance times for a fluid element within the model pooled secretions have been calculated based on the geometry of the system and the rate of leakage data illustrated in 1, and are presented below A longer resonance time is significant because it allows for: 1) more time for the fluid to be removed (i.e., suctioned) from above the cuff before being aspirated by the patient if intermittent suctioning is being used, and; 2) less aggressive suctioning being needed if continuous suction is being used. These resulting synergies are compared quantitatively to a conventional ET tube in FIG. 2.

C. Synergies in Combining Improved Sealing Technology with an Eluting Antimicrobial Coating

1. Synergies Above the ET Tube Cuff

The residence or resonance time calculated above, which allots care givers more time between suctioning and cleaning events, can also be thought of as a “contact time” between the fluid and the outside wall of the ET tube. All else being equal, a longer contact time between a fluid and a surface coated with an active ingredient designed to elute into the fluid environment results in more of the active ingredient being eluted per unit volume of fluid. A higher concentration of active ingredient per unit volume usually means 1) the influence of the active will be more pronounced or 2) the concentration of the coating applied to the surface can be reduced to achieve the same level of activity.

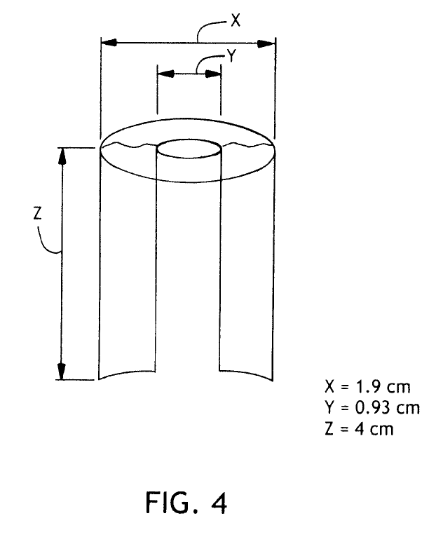

To test this hypothesis, the following system was used as an example. A piece of ET tube surface, coated with an antimicrobial technology based on eluting ionic silver, was challenged with a known concentration of bacteria. Starting concentrations of bacteria were chosen to be representative of that in the oral cavity of a healthy patient. The ratio of coated surface area to fluid volume was based upon the geometry of the model system described above. The coated piece was allowed to interact with the fluid media for various times, based upon the “contact” times calculated in FIG. 4. Results are presented in FIG. 5. As seen from this figure, the longer contact times, those representative of the present invention, resulted in greater reduction of bacteria that reside in the surrounding fluid. At higher initial concentrations of bacteria similar trends hold, until a point is reached where the dynamics of bacterial growth overwhelm the eluted active. This point will differ depending upon the overall dynamics of the specific system of interest. In the case of the present invention, combining an eluting coating on the extra-luminal surface proximal to the cuff with an improved sealing cuff technology, resulted in a synergistic effect; the synergistic effect being 1) longer contact time of the coated surface with contaminated fluid pooled proximal to the cuff and 2) greater bacterial reduction at certain points in time, as presented in FIG. 5.

It should be noted the trends illustrated in FIG. 5 are independent of the specific improved sealing cuff technology used and that any improved sealing technology resulting in similar contact times would be expected to result in similar synergies when combined with an eluting coating. In this example, the lower rate of leakage and resulting longer contact time is explained by the difference in fold structure and dimensions of the cuff balloon as compared to competition.

2. Synergies within Cuff Folds

According to an aspect of the invention, the synergies described herein better manifest when using desirably high volume/low pressure cuffs, for example those as described by F. Göbel. Conventional low volume/high pressure cuffs, as well as low volume/low pressure cuffs such as described by Young et al (above), may be employed but are less preferred. A low-volume, low-pressure tracheal tube cuff reduces pulmonary aspiration. It was hypothesized that structural differences in the smaller diameter channels and fold geometry would result in more fold surface area per fold channel volume. To test this hypothesis, the geometry of cuff folds were measured via micro-computerized tomography (CT) scans using a Skyscan 1172a Micro-CT x-ray instrument. Estimates of how much higher this surface area to volume ratio (SA/V) is within the present cuff folds vs. competitors were calculated from reconstructions analyzed using Skyscan CTAnalyzer (v. 1.02, Build 35) programming to obtain the desired dimensional measurements. Data are given in Table 2 and Table 3 below. Contact times are also given, based on the leakage data presented above and the calculated amount of time it takes for fluid to traverse an average size fold from the top of the cuff to the bottom of the cuff. Note that two sets of data are presented; the difference being in whether the cuff folds were “random” in nature or more aligned parallel to the tube axis by means of a pre-aligning step to align the channels.

TABLE 2
Random Channels

| | | Present | Comparison | |
|---|---|---|---|---|
| | Comparison B | Invention | A | |
| Z contact length | 31.5 | 34.5 | 24 | mm |
| Avg x-sect A | 2.5 | 0.7 | 5.7 | mm2 |
| Avg Void V | 79 | 26 | 136 | mm3 |
| Avg Total Per | 25 | 11 | 40 | mm |
| Avg Surface A | 793 | 396 | 949 | mm2 |
| Surface A/V | 10 | 15 | 7 | 1/mm |
| | 100 | 154 | 70 | 1/cm |
| % greater | 54 | | | |
| % greater | | 121 | | |
| Leak Rate | 3.14 | 0.02 | N/A | cm3/min |
| Avg Void V | 79 | 26 | 136 | mm3 |
| Con Time | 1.5 | 81 | N/A | sec |
| | 0.0 | 1.3 | N/A | min |
| | 0.00 | 0.02 | N/A | hr |

| | |
|---|---|
| % greater | 5246 |

| | |
|---|---|
| % greater | >5246 |

TABLE 3
Aligned Channels

| | | Present | Comparison | |
|---|---|---|---|---|
| | Comparison B | Invention | A | |
| Z contact length | 31.5 | 34.5 | 24.0 | mm |
| Avg x-sect A | 1.0 | 0.1 | 1.5 | mm2 |
| Avg Void V | 32.2 | 2.5 | 36.0 | mm3 |
| Avg Total Per | 13.4 | 3.4 | 18.9 | mm |
| Avg Surface A | 423 | 116 | 455 | mm2 |
| Surface A/V | 13.1 | 45.8 | 12.6 | 1/mm |
| | 131 | 458 | 126 | 1/cm |
| % greater | 249 | | | |
| % greater | | 262 | | |
| Leak Rate | 0.440 | 0.002 | N/A | cm3/min |
| Avg Void V | 32 | 3 | 36 | mm3 |
| Contact Time | 4.4 | 76 | N/A | sec |
| | 0.07 | 1.3 | N/A | min |
| | 0.00 | 0.02 | N/A | hr |

| | |
|---|---|
| % greater | 1627 |

| | |
|---|---|
| % greater | >1627 |

In comparing the two tables above, regardless of whether the channels produced by the cuff foils were more aligned or more random in nature, the cuff design of the present invention resulted in a calculated greater surface area to volume (SA/V) ratio, substantiating the hypothesis above. Moreover, the SA/V ratio was surprisingly greater than the nearest competitor by 54% and 249%, respectively, depending upon whether the channels were random or more aligned in nature. These percentages were calculated based on the following ratio:

[(SA/V ratio of cuff balloon)−(SA/V ratio of comparative device)]/(SA/V ratio of comparative device)×100

If the ET cuff surface were to be coated with an eluting coating, it is hypothesized that the increased surface area to volume ratio should provide improved efficacy. To test the hypothesis that the higher SA/V ratios would improve microbial efficacy, the SA/V ratios, estimated above when cuff channels were aligned, were utilized in a bench top protocol. Within this protocol SA/V ratios having a similar disparity, but not the same absolute values, were tested. In particular, testing of large SA/V ratios (˜100) in vitro presents challenges. As such, for this work, smaller SA/V ratios were used to test for differences in antimicrobial performance; however, the percent differences were chosen to model the differences between the present inventive cuff design and the comparative design. Specifically, 1.6 cm²/ml and 4.0 cm²/ml were used for the conventional and the inventive design, respectively, versus the 131 cm²/ml and 458 cm²/ml calculated from the CT images above. After 4 hours of contact time, the greater SA/V ratio began to differentiate itself in terms of antimicrobial performance, as presented graphically in FIG. 5 and statistical analysis presented in Table 4. This increase in reduction of CFU/ml substantiates the hypothesis that the increased SA/V ratio, within the folds resulting from the present invention, results in a synergistic effect in using an eluting coating in combination with improved cuff sealing technology; the improved cuff sealing technology being a foil membrane as described by F. Göbel in this embodiment.

TABLE 4
t-Test: Two-Sample Assuming Unequal Variances

| Present | | | Present | Comparison |
|---|---|---|---|---|
| Invention | Comparison B | | Invention | B |
| 8.5E+02 | 3.5E+03 | Mean | 1.4E+03 | 2.9E+03 |
| 1.7E+03 | 3.3E+03 | Variance | 2.3E+05 | 6.8E+05 |
| 1.7E+03 | 2.0E+03 | Observations | 3 | 3 |
| | | Hypothesized | 0 | |
| | | Mean D | | |
| | | df | 3 | |
| | | t Stat | −2.724 | |
| | | P(T <= t) one-tail | 0.036 | |
| | | t Critical one-tail | 2.353 | |
| | | P(T <= t) two-tail | 0.072 | |
| | | t Critical two-tail | 3.182 | |

Moreover, as tabulated in Table 2 and 3, the lower rate of leakage measured, caused by the difference in fold geometry, results in greater “resonance time” of the fluid within the folds. As above, this resonance time can also be viewed as a “contact time” between the coated cuff surface and the fluid passing through the folds. This would also be considered to contribute to additional synergistic antimicrobial performance. However, it should be recognized the contact times are much shorter within the cuff folds as compared to above the cuff (˜seconds to traverse to folds vs. minutes or hours to progress through about 11 ml pool of liquid).

It is also worth noting, while the degree to which the present invention cuff SA/V ratio exceeds the competition decreases in going from aligned channels to random channels (i.e., decreases from 249%-aligned to 54%-random), the disparity between contact times increases (i.e., increases from 1627%-aligned to 5246%-random) through a more pronounced reduction in rate of leakage when using the present invention cuff technology. This serendipitous result should lead to a more “robust” technology when combining an eluting coating with the present cuff technology. That is, if channels were to progress over time from being more aligned to more random, reduction in SA/V could be partially compensated for by increasing contact time.

Finally, while the examples above involve the application of antimicrobial silver nanotechnology (from AcryMed Inc.) to an ET tube, the synergies demonstrated are generic to all eluting coatings, antimicrobial or not (e.g., analgesic, anti-thrombotic, etc.) and would be important in any medical device application where mass transport is a governing factor in resulting efficacy.

Other structural differences exist between the present cuff design and the designs of Comparison A and B, which are summarized in Table 5, below. Of particular note are the comparative differences in total length of model tracheal wall not in contact with cuff material; i.e., there is more than twice the perimeter “length” of tracheal wall not in contact with cuff material when using competitive technologies vs. with the present cuff design. Conversely, with the present cuff design there is more cuff circumference in contact with the model tracheal circumference. This, coupled with a smaller cuff circumference used for the present ET tube design, equates to less total length of the cuff balloon participating in a fold.

According to the present invention, the cuff bladder has a ratio of the total length of a cuff wall-surface length that does not contact a tracheal wall versus the total length of a cuff wall-surface length that does contact said tracheal wall of about 22% or less, typically about 20% or less, more typically about 17% or less, and desirably about 15% or less. As the example illustrates, the ratio is less than 10%.

The cuff balloon exhibits a total cuff wall-surface length to fold length ratio of about 0.5.

TABLE 5

| | | Present | |
|---|---|---|---|
| Comparison B | Comparison A | Invention | |
| 11.8 | 13.5 | 4.2 | Total length of wall not in |
| | | | contact with cuff |
| 41.6 | 39.9 | 49.2 | Total cuff length in contact |
| | | | with cylinder wall |
| 84.8 | 94.4 | 69.1 | Circumference of cuff |
| 43.2 | 54.5 | 19.9 | Total cuff length |
| | | | participating in a fold |

*7 mm ET tube, 25 cm H2O pressure, all values in mm.

Taking the average length of tracheal wall not contacted by the cuff per fold, shown in Table 6, below, the present inventive design fits and conforms better on average to the surface when a fold occurs, such as illustrated in FIG. 7.

TABLE 6

| Example | Length/Fold (mm) |
|---|---|
| Comparison A | 1.30 |
| Comparison B | 1.48 |
| Present Invention | 0.50 |

The present invention has been described both in general and in detail by way of examples. Persons skilled in the art will understand that the invention is not limited necessarily to the specific embodiments disclosed. Modifications and variations may be made without departing from the scope of the invention as defined by the following claims or their equivalents, including equivalent components presently known, or to be developed, which may be used within the scope of the present invention. Therefore, unless changes otherwise depart from the scope of the invention, the changes should be construed as being included herein.

We claim:

1. A method of caring for an intubated patient, the method comprising: providing an endotracheal tube having a cannula and an inflatable cuff bladder in which at least a portion of the cannula or the cuff bladder has either an eluting or erodable active coating for delivering a medicinal or antimicrobial agent; applying said endotracheal tube to said patient and inflating said cuff bladder to a pressure of between about 10 cm H₂O to about 30 cm H₂O; letting said endotracheal tube remain inflated within said patient for a period of more than about 30-40 minutes; and removing accumulated fluids from a zone in said patient's respiratory passage above said cuff bladder at maximum once within a period of at least one hour.

2. The method according to claim 1, wherein said removal of accumulated fluids is performed at maximum once within a period of 1.5 to 2 hours.

3. The method according to claim 1, wherein said removal of accumulated fluids is performed at maximum once within a period of greater than 2 hours.

4. The method according to claim 1, wherein said endotracheal tube and inflated cuff bladder retard leakage of a volume of accumulated fluids from a zone above or proximal to said cuff bladder into said patient's lower respiratory passages for at least one hour.

5. The method according to claim 1, wherein said endotracheal tube and inflated cuff bladder retard leakage of a volume of accumulated fluids from a zone above or proximal to said cuff bladder into said patient's lower respiratory passages for 2-3 hours.

6. The method according to claim 5, wherein said volume of accumulated fluids is slowed from leaking by a cuff bladder inflation pressure of less than about 20-18 cm H₂O.

7. The method according to claim 1, wherein said endotracheal tube and inflated cuff bladder retard leakage of a volume of accumulated fluids from a zone above said cuff bladder to said patient's lower respiratory passages for over 3 hours.

8. The method according to claim 1, wherein said endotracheal tube and inflated cuff bladder retard leakage of a volume of accumulated fluids from a zone above or proximal to said cuff bladder into said patient's lower respiratory passages for a period of up to about 7 hours at a cuff bladder inflation pressure of no greater than about 25-28 cm H₂O.

9. The method according to claim 1, wherein said endotracheal tube has integrated a separate suction lumen integrated into the wall to facilitate removal via suction accumulated fluids.

10. The method according to claim 1, wherein said endotracheal tube and cuff bladder each have a surface that is at least partially treated with an eluting coating containing an antimicrobial, antibiotic or antiseptic active reagent.

11. The method according to claim 10, wherein said antimicrobial, antibiotic or antiseptic active reagent neutralizes, inactivates, or kills pathogenic microbes within said accumulated fluids.

12. The method according to claim 1, wherein said endotracheal tube and cuff bladder each have a surface coating that is erodable in an aqueous environment.

13. A medical device comprising: an inflatable bladder attached co-axially around a hollow conduit; a surface coating which provides a extended-time controlled-release source for reactive or therapeutic agents and is situated over at least part of a surface of said bladder, said bladder being inflated restrictively within a channel so that any fluid flowing through the channel, but external to said hollow conduit, fully interacts with the reactive or therapeutic agents.

14. The medical device according to claim 13, wherein the eluting surface coating covers at least part of an outer surface of said hollow conduit.

15. The medical device according to claim 13, wherein at least part of said hollow conduit or said bladder has a coating that forms either an eluting or erodable surface when exposed for a predetermined period in an aqueous environment.

16. The medical device according to claim 13, wherein said bladder has an inflation pressure of between about 10 cm H₂O and less than about 30 cm H₂O.

17. The medical device according to claim 13, wherein said inflatable bladder retards leakage of a volume of accumulated fluids from a zone above said bladder into a patient's lower respiratory passages for over 3 hours at a bladder inflation pressure of between less than about 22 cm H₂O.

18. The medical device according to claim 13, wherein said inflatable bladder retards leakage of a volume of accumulated fluids from a zone above or proximal to said cuff bladder into said patient's lower respiratory passages for a period of up to about 7 hours at a bladder inflation pressure of no greater than about 25-28 cm H₂O.

19. The medical device according to claim 13, wherein said hollow conduit has integrated a separate suction lumen integrated into the wall to facilitate removal via suction accumulated fluids.

20. The medical device according to claim 13, wherein said hollow conduit and bladder each have a surface that is at least partially treated with an eluting coating containing an antimicrobial, antibiotic or antiseptic active reagent.

21. The medical device according to claim 20, wherein said antimicrobial, antibiotic or antiseptic active reagent neutralizes, inactivates, or kills pathogenic microbes within said accumulated fluids.

22. An airway management device comprising: a cuffed balloon that obturates a patient's trachea below said patient's glottis, an air tube, the cuffed balloon being attached to said air tube and having a size larger than a tracheal diameter when in a fully inflated state and being made of a soft, flexible membrane material, said cuffed balloon having at least a portion with an eluting surface coating that serves as a controlled-release reservoir of antimicrobial, antibiotic or antiseptic agents or therapeutic agents, and when used in said patient's trachea said cuffed balloon is adapted to retard leakage of a volume of fluid from a zone above said cuff bladder to said patient's lower respiratory passages for a period of more than about 30-40 minutes at a cuff bladder inflation pressure of between about 10 cm H₂O to about 30 cm H₂O.

23. The airway management device according to claim 22, wherein said cuff bladder is adapted to retard leakage of a volume of fluid from a zone above said cuff bladder to said patient's lower respiratory passages for at least one hour.

24. The airway management device according to claim 22, wherein said cuff bladder is adapted to retard leakage of a volume of fluid from a zone above said cuff bladder to said patient's lower respiratory passages for about 120 to about 180 minutes at an inflation pressure of less than about 20 cm H₂O.

25. The airway management device according to claim 22, wherein said cuff bladder is adapted to retard leakage of a volume of fluid from a zone above said cuff bladder to said patient's lower respiratory passages for over 180 minutes at an inflation pressure of no greater than about 25-28 cm H₂O.

26. The airway management device according to claim 22, wherein said air tube and said cuff balloon each has a surface with either an eluting or erodable coating.

27. The airway management device according to claim 22, wherein said cuff bladder has a ratio of the total length of a cuff wall-surface length that does not contact a tracheal wall versus the total length of a cuff wall-surface length that does contact said tracheal wall of about 22% or less.

28. The airway management device according to claim 27, wherein said a ratio of said wall-surface length that does not contact a tracheal wall versus a cuff wall-surface length that does contact said tracheal wall of less than about 20%.

29. The airway management device according to claim 22, wherein said cuff balloon exhibits a total cuff wall-surface length to fold length ratio of about 0.5.

30. An endotracheal tube comprising: a cuff balloon situated around a hollow conduit, said cuff balloon and hollow conduit each having on at least a portion of its surface a controlled-release coating of an antimicrobial or antiseptic agent, and when in a patient's trachea said cuffed balloon retards leakage of a volume of accumulated fluids from a zone above or proximal to said cuffed bladder into said patient's lower respiratory passages for a period of up to about 7 hours at a bladder inflation pressure of no greater than about 30 cm H₂O.

31. The endotracheal tube according to claim 30, wherein said cuff balloon retards leakage for a period of at least about 120 minutes to about 180 minutes at an inflation pressure of about 15-17 cm H₂O.

32. The endotracheal tube according to claim 30, wherein said cuff bladder has a ratio of the total length of a cuff wall-surface length that does not contact a tracheal wall versus the total length of a cuff wall-surface length that does contact said tracheal wall of about 22% or less.

33. The airway management device according to claim 32, wherein said a ratio of said wall-surface length that does not contact a tracheal wall versus a cuff wall-surface length that does contact said tracheal wall of about 15% or less.

34. The airway management device according to claim 30, wherein said cuff balloon exhibits a total cuff wall-surface length to fold length ratio of about 0.5.

35. A method of instructing a caregiver, the method comprising: providing instruction to a healthcare worker on the use of an endotracheal tube with an inflatable cuff bladder in which at least a portion of the endotracheal tube or cuff bladder has an active coating for delivering a medicinal or antimicrobial agent, and a cuff inflation pressure of between about 10 cm H₂O to about 30 cm H₂O for a predetermined in-dwelling duration of at least about 120 minutes or more; and either removing or not removing accumulated fluids from a zone in said patient's respiratory passage above the cuff bladder at maximum once within a period of at least two hours.
